(19) United States
(12) Patent Application Publication
(10) Pub. No.: US 2011/0270396 A1
(43) Pub. Date: Nov. 3, 2011

(54) EXPANDABLE IMPLANTS FOR STABILIZING ADJACENT ANATOMICAL STRUCTURES

(75) Inventor: Carla Leibowitz, San Carlos, CA (US)

(73) Assignee: Warsaw Orthopedic, Inc, Warsaw, IN (US)

(21) Appl. No.: 12/770,446

(22) Filed: Apr. 29, 2010

(51) Int. Cl. A61F 2/44

(52) U.S. Cl. 623/17.11

(57) ABSTRACT

In one form, an expandable implant configured for positioning between one or more adjacent bones or bony portions, such as vertebrae of the spinal column for example, is provided. In one aspect, the implant includes a plurality of deformable members that is each positionable between an unexpanded configuration and an expanded configuration in response to axial compression of the deformable members. In the unexpanded configuration, each of the deformable members engages with one or more immediately adjacent deformable members, while in the expanded configuration each of the deformable members further engages with one or more additional deformable members positioned on the other side of the one or more immediately adjacent deformable members. In another form, a method for positioning an expandable implant in a minimally invasive approach is provided. However, in other embodiments, different forms and applications are envisioned.

### BACKGROUND

The present invention is generally directed to implants and devices for positioning between one or more adjacent bones or bony portions, such as one or more vertebral bodies of the spinal column.

The repair, reconstruction and stabilization of bony structures is sometimes accomplished by directly fixing adjacent bony structures to each other, such as by a plate. Another type of stabilization technique uses one or more elongated rods extending between components of a bony structure and secured to the bony structure to stabilize the components relative to one another. In other instances, bone growth inducing material can be introduced between the adjacent bony structures, which over time results in a solid bony connection. In some instances, the adjacent bony structures are not sufficiently strong to maintain their patency as the bone heals or the bone grows between the adjacent structures through the bone growth inducing material. In these instances, one or more mesh structures, spacers or cages have been provided to engage the adjacent bony structures to provide additional stability. In one or more forms, these devices may be hollow and can be configured to contact the harder cortical bone of the adjacent bony structures.

In some instances, the placement of one or more of these devices between the adjacent bony structures involves cutting, removing, and or repositioning skin and tissue surrounding the surgical site in order for the surgeon to access the location where the stabilization device is to be installed. This type of approach to the surgical site may cause trauma, damage, and scarring to the tissue, and also presents risks that the tissue will become infected and that a long recovery time will be required after surgery for the tissue to heal.

Minimally invasive surgical techniques are particularly desirable in, for example, spinal and neurosurgical applications because of the need for access to locations deep within the body and the presence of vital intervening tissues. The development of percutaneous minimally invasive spinal procedures has yielded a major improvement in reducing recovery time and post-operative pain because they require minimal, if any, muscle dissection and can be performed under local anesthesia. These benefits of minimally invasive techniques have also found application in surgeries for other locations in the body where it is desirable to minimize tissue disruption and trauma. However, there remains a need for further improvements in implants, devices, instruments, systems and methods for stabilizing bony structures using minimally invasive and other techniques.

### SUMMARY

In one nonlimiting embodiment of the present application embodiment, an implant or device is configured to provide stability and spacing between adjacent bones or bony structures.

In another embodiment, an expandable implant or device is configured for positioning at a location between one or more adjacent bones or bony structures, such as vertebrae of the spinal column for example. In one aspect, the implant includes a plurality of deformable members that is each positionable between an unexpanded configuration and an expanded configuration in response to axial compression of the deformable members. The deformable members are positioned about an elongated shaft and the implant may be positioned at the location between the adjacent bones with the deformable members in the unexpanded configuration. An axial compression force may then be applied to the deformable members in order to expand the deformable members from the unexpanded configuration to the expanded configuration. In one aspect, the positioning of the implant at the location between the adjacent bones and the application of the axial compression force to expand the deformable members to the expanded configuration are performed through a minimally invasive surgical approach, although other approaches are also contemplated. However, in other embodiments, different forms and applications are envisioned.

For example, in one other embodiment, an expandable implant includes a plurality of deformable members that is each positionable between an unexpanded configuration and an expanded configuration in response to axial compression of the deformable members. In the unexpanded configuration, each of the deformable members engages with one or more immediately adjacent deformable members, while in the expanded configuration each of the deformable members further engages with one or more additional deformable members positioned on the other side of the one or more immediately adjacent deformable members.

In yet another embodiment, an implant includes a body including a plurality of deformable members positionable between an unexpanded configuration and an expanded configuration in response to axial compression. Moreover, at least a first member of the plurality of deformable members engages with a second member of the plurality of deformable members when each of the first and second members is in the unexpanded configuration, and the first member further engages with a third member of the plurality of deformable members when each of the first, second and third members is in the expanded configuration. In addition, the second member is positioned between the first member and the third member.

In another embodiment, an implant includes an elongated shaft and a plurality of deformable members positioned about the elongated shaft. Each of the plurality of deformable members includes a body positionable between an unexpanded configuration and an expanded configuration. The body also includes oppositely positioned first and second ends and first and second sidewalls extending between the first and second ends. In the unexpanded configuration the body of each of the plurality of deformable members is elongated along the elongated shaft. However, in the expanded configuration the body of each of the plurality of deformable members is elongated in a direction that extends transversely to the elongated shaft and oppositely positioned first and second portions of each of the first and second sidewalls extend obliquely away from the elongated shaft and one another.

Other embodiments of the present application are directed to methods and techniques for positioning one or more expandable implants in a disc space between adjacent vertebrae of the spinal column.

Another embodiment of the present application is a unique device for positioning between and stabilizing adjacent bones or bony structures. Other embodiments include unique methods, systems, devices, kits, assemblies, equipment, and/or apparatus involving the stabilization and support of adjacent bones or bony structures.

Further embodiments, forms, features, aspects, benefits, objects and advantages of the present application shall become apparent from the detailed description and figures provided herewith.

### BRIEF DESCRIPTION OF THE FIGURES

FIG. 1 is a perspective view of an expandable implant in an unexpanded configuration.

FIG. 2 is an enlarged, perspective view of a deformable member of the implant illustrated in FIG. 1 in an unexpanded configuration.

FIG. 3A is a perspective view of the implant illustrated in FIG. 1 in an expanded configuration.

FIGS. 3B and 3C are end and side plan views, respectively, of the implant illustrated in FIG. 1 in an expanded configuration.

FIG. 4 is a perspective view of the deformable member illustrated in FIG. 2 in an expanded configuration.

FIG. 5 is a perspective view of an alternative embodiment expandable implant in an unexpanded configuration.

FIG. 6 is a perspective view of the implant illustrated in FIG. 5 in an expanded configuration.

FIGS. 7-10 illustrate various steps of a minimally invasive surgical procedure for inserting the expandable implant illustrated in FIG. 1 or FIG. 5.

FIG. 11 is an end, plan view of the implant of FIG. 1 positioned between adjacent bony structures.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

The subject application is generally directed to implants configured to provide stability and spacing between adjacent bones or bony structures, such as adjacent vertebrae of the spinal column, although use of the implants described herein at locations other than the spinal column are also contemplated. In one embodiment, an expandable implant or device is configured for positioning at a location between one or more adjacent bones or bony structures to provide stability and/or spacing between the adjacent bones. In one aspect, the implant includes a plurality of deformable members that is each positionable between an unexpanded configuration and an expanded configuration in response to axial compression of the deformable members. The deformable members are positioned about an elongated shaft and the implant may be positioned at the location between the adjacent bones with the deformable members in the unexpanded configuration. An axial compression force may then be applied to the deformable members in order to expand the deformable members from the unexpanded configuration to the expanded configuration. Still, it should be appreciated that alternative forms, aspects, configurations, arrangements and methods are contemplated with respect to the subject matter disclosed and described herein.

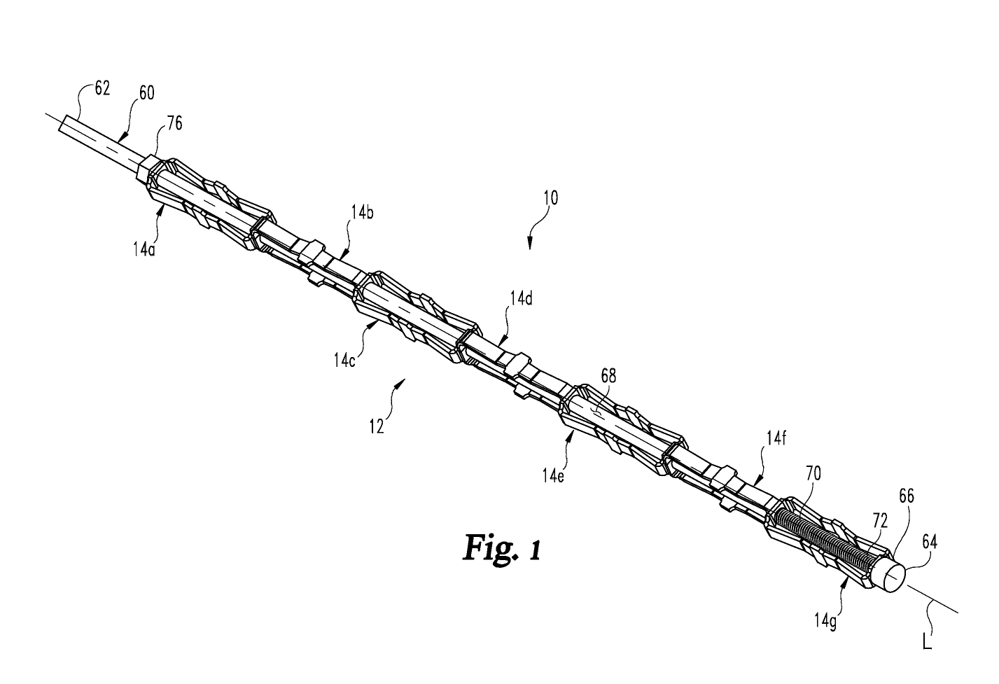

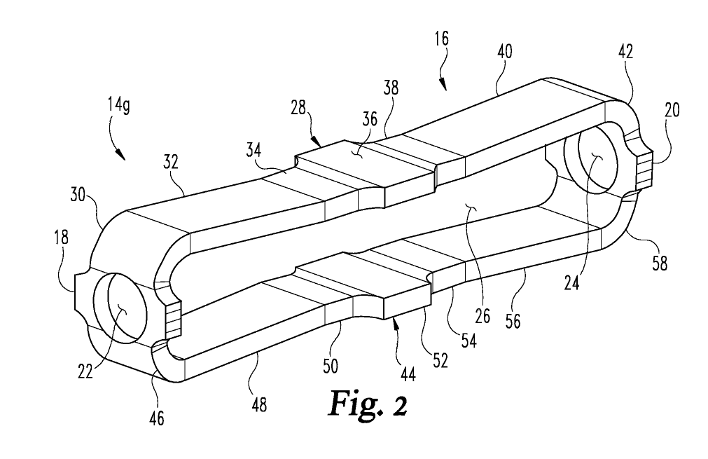

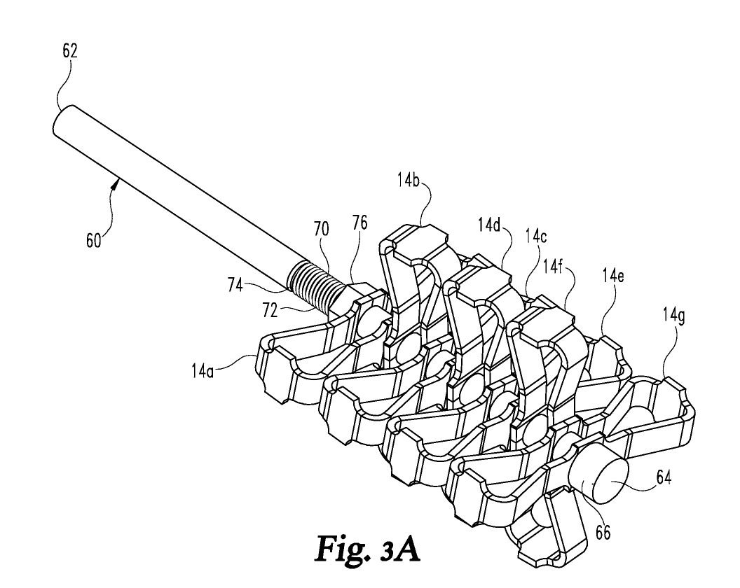

Referring now to FIG. 1, there is shown an expandable implant 10 in an unexpanded configuration. Implant 10 includes a plurality of deformable members 14a-14g positioned on an elongated shaft 60. Deformable members 14a-14g are each positionable between an unexpanded configuration, as illustrated in FIGS. 1 and 2 for example, and an expanded configuration, as illustrated in FIGS. 3A-C and 4 for example. With reference to FIG. 2 for example, further details of deformable member 14g in an unexpanded configuration will be provided. It should be appreciated however that the description provided in connection with deformable member 14g is also generally applicable to deformable members 14a-14f. Deformable member 14g includes an elongate body 16 that extends between a proximal end 18 and a distal end 20. Proximal end 18 includes an aperture 22 extending therethrough and distal end 20 includes an aperture 24 extending therethrough. Apertures 22 and 24 communicate with a hollow interior 26 situated between oppositely positioned sidewalls 28 and 44 that extend between proximal end 18 and distal end 20. Sidewall 28 includes a first portion 30, a second portion 32, a third portion 34, a fourth portion 38, a fifth portion 40, a sixth portion 42 and an intermediate portion 36 positioned between portions 30, 32 and 34 and portions 38, 40 and 42. As illustrated in FIGS. 1 and 2 for example, intermediate portion 36 is wider than and extends laterally beyond portions 30, 32, 34, 38, 40 and 42. Moreover, in the unexpanded configuration of deformable member 14g, portions 30 and 42 are convexly rounded adjacent to proximal end 18 and distal end 20, respectively, and portions 34 and 38 adjacent to intermediate portion 36 have a generally linear configuration and are slightly angled relative to portion 32 and intermediate portion 36 and portion 40 and intermediate portion 36, respectively.

Similar to sidewall 28, sidewall 44 includes a first portion 46, a second portion 48, a third portion 50, a fourth portion 54, a fifth portion 56, a sixth portion 58 and an intermediate portion 52 positioned between portions 46, 48 and 50 and portions 54, 56 and 58. As illustrated in FIGS. 1 and 2 for example, intermediate portion 52 is wider than and extends laterally beyond portions 46, 48, 50, 54, 56 and 58. Moreover, in the unexpanded configuration of deformable member 14g, portions 46 and 58 are convexly rounded adjacent to proximal end 18 and distal end 20, respectively, and portions 50 and 54 adjacent to intermediate portion 52 have a generally linear configuration and are slightly angled relative to portion 48 and intermediate portion 52 and portion 56 and intermediate portion 52, respectively.

Elongated shaft 60 extends along a longitudinal axis L between a proximal end 62 and a distal end 64 and includes a first portion 68 including proximal end 62 and a second portion 70 including distal end 64. In the illustrated form, first and second portions 68 and 70 are releasably coupled together by a frangible portion 74 (shown in FIG. 3A). More particularly, upon the application of sufficient rotational torque on first portion 68 relative to second portion 70, first portion 68 will sever from second portion 70 at frangible portion 74. In other non-illustrated forms however, alternative arrangements for releasably coupling first portion 68 and second portion 70 are contemplated. For example, in one form, one of first portion 68 and second portion 70 can be provided with an internally threaded receptacle that receives and engages with an externally threaded portion of the other of first portion 68 and second portion 70 to provide a releasable coupling between first portion 68 and second portion 70. In another form, it is contemplated that first portion 68 and second portion 70 can be releasably coupled by a ball-detent mechanism or releasably interlocking cams and tabs, just to provide a few possibilities.

Second portion 70 also includes an enlarged flange or head portion 66 that extends proximally from distal end 64 to external threading 72 positioned around the external surface of second portion 70. In the illustrated form, first portion 68 is free from external threading, although forms of elongated shaft 60 where first portion 68 includes external threading are contemplated. A compression member 76 is positioned on elongated shaft 60 adjacent to proximal end 62 such that deformable members 14a-14g are positioned between compression member 76 and flange portion 66. Compression member 76 may be in the form of an internally threaded nut for example and include an internally threaded passage. In one form, compression member 76 can be configured such that it is axially slidable, without rotation, along the exterior surface of first portion 68 of elongated shaft 60 until it reaches and engages with external threading 72 of second portion 70. For example, it is contemplated that the diameter of the internally threaded passage of compression member 76 may be greater than the diameter of first portion 68 of elongated shaft 60. Once the internal threading of compression member 76 engages with external threading 72 on second portion 70 of elongated shaft 60 however, compression member 76 must be rotated in order to be axially moved along second portion 70.

In the unexpanded configuration of implant 10, deformable members 14a-14g are generally positioned in an end-to-end arrangement along elongated shaft 60. Similarly, the distal end of each of deformable members 14a-14f engages with the proximal end of the adjacent, distally positioned respective one of deformable members 14b-14g. Moreover, in the unexpanded configuration, deformable members 14a and 14g only engage with or contact one other deformable member while each of deformable members 14b-14f engages with or contacts two other deformable members; i.e., those deformable members positioned proximally and distally thereof. In the illustrated embodiment, the sidewalls of each of deformable members 14a-14g are axially offset about elongated shaft 60 relative to the sidewalls of any adjacent deformable member. More particularly, the sidewalls of deformable member 14b are rotated about ninety degrees about elongated shaft 60 relative to the sidewalls of deformable members 14a and 14c positioned on opposite sides thereof. Furthermore, deformable members 14a-14g are rotatable about elongated shaft 60 but are coupled together in an arrangement that prevents rotational movement of deformable members 14a-14g about elongated shaft 60 relative to one another. For example, in one form, it is contemplated that the ends of deformable members 14a-14g could be welded or otherwise fused together. In another form however, it is contemplated that the ends of deformable members 14a-14g could be releasably coupled together by a ball-detent, pin-aperture or other releasable arrangement that prevents rotation of deformable members 14a-14g about elongated shaft 60 relative to one another. In other forms, it is contemplated that deformable members 14a-14g may be non-rotatably positioned about elongated shaft 60 and relative to one another. For example, in one non-illustrated form, implant 10 could be provided with a keyed configuration between elongated shaft 60 and deformable members 14a-14g. More particularly, in one such form, elongated shaft 60 can include a square, rectangular, triangular, or star shaped cross-sectional configuration, just to provide a few possibilities, and the apertures in the proximal and distal ends of deformable members 14a-14g can be correspondingly configured such that engagement between the apertures and elongated shaft 60 prevents rotation of deformable members 14a-14g about elongated shaft 60 and relative to one another.

While not previously discussed, it should be appreciated that implant 10 can be provided with one or more deformable members in addition to deformable members 14a-14g. In addition, it is also contemplated that implant 10 could be provided with fewer deformable members than what is shown in the illustrated embodiment. For example, in one form, it is contemplated that implant 10 could include two or more deformable members; although a form where implant 10 only includes a single deformable member is also contemplated. Moreover, in the form where the ends of deformable members 14a-14g are releasably coupled to one another, it is contemplated that compression member 76 could be removed from elongated shaft 60 to allow removal of one or more of deformable members 14a-14g in order adjust the overall length of implant 10. Similarly, in one form, a kit could include a plurality of differently sized and/or shaped deformable members that can be positioned on the elongated shaft 60 such that a surgeon can custom design implant 10 for use at a variety of anatomical locations or to address one or more conditions encountered during surgery.

In one manner of expanding deformable members 14a-14g from their unexpanded configuration to their expanded configuration, compression member 76 may be moved distally along elongated shaft 60 toward distal end 64. Flange portion 66 prevents distal movement of deformable members 14a-14g such that deformable members 14a-14g become axially compressed as compression member 76 is moved distally along elongated shaft 60. In another approach, it is contemplated that a proximal pulling force could be applied to elongated shaft 60 in addition to or in lieu of distal movement of compression member 76 in order to axially compress deformable members 14a-14g positioned between compression member 76 and flange portion 66. Further details regarding the axial compression and expansion of deformable members 14a-14g to their expanded configuration are provided in connection deformable member 14g. It should be appreciated however that the description provided in connection with deformable member 14g with respect to its conversion from its unexpanded configuration to its expanded configuration is also generally applicable to deformable members 14a-14f.

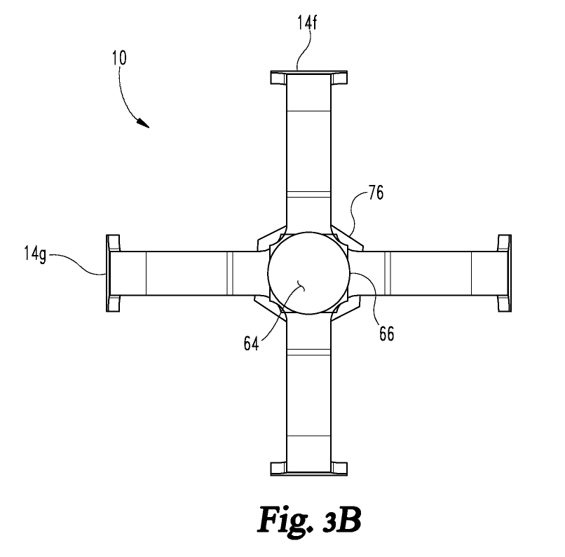

As axial compression is applied to deformable member 14g, proximal end 18 and distal end 20 are brought together and sidewalls 28 and 42 become laterally displaced relative to elongated shaft 60. More particularly, with respect to sidewall 28, portions 34 and 38 begin to bend as deformable member 14g is axially compressed to allow lateral displacement of intermediate portion 36 from elongated shaft 60. In addition, portions 30 and 42 also begin to straighten as deformable member 14g is axially compressed. With respect to sidewall 44, portions 50 and 54 begin to bend as deformable member 14g is axially compressed to allow lateral displacement of intermediate portion 52 from elongated shaft 60. In addition, portions 46 and 58 also begin to straighten as deformable member 14g is axially compressed. As illustrated in FIG. 4 for example where deformable member 14g is shown in the expanded configuration, axial compression can be applied to deformable member 14g until proximal end 18 and distal end 20 are positioned adjacent to one another and deformable member 14g becomes elongated in a direction that extends transversely to elongated shaft 60. In the illustrated form, a space remains between proximal end 18 and distal end 20 when deformable member 14g is in the expanded configuration. However, in an alternative form, it is contemplated that proximal end 18 and distal end 20 could be positioned against one another when deformable member 14g is in the expanded configuration such that no space remains therebetween.

Once deformable member 14g is in the expanded configuration, portions 34 and 38 generally become convexly curved and portions 30 and 42 take on a generally straighter configuration relative to their shape in the unexpanded configuration of deformable member 14g. Similarly, portions 50 and 54 generally become convexly curved while portions 46 and 58 take on a generally straighter configuration relative to their shape in the unexpanded configuration of deformable member 14g. In addition, in the expanded configuration of deformable member 14g portions 32 and 40 generally extend obliquely away from elongated shaft 60 and from one another. Portions 48 and 56 also generally extend obliquely away from elongated shaft 60 and form one another when deformable member 14g is in the expanded configuration. In view of the foregoing, it should be appreciated that portions 30, 34, 38 and 42 of sidewall 28 and portions 46, 50, 54 and 58 of sidewall 44 provide relief portions which facilitate the conversion of deformable member 14g from the unexpanded configuration to the expanded configuration. However, it should be appreciated that one or more other portions of deformable member 14g in addition to or in lieu of portions 30, 34, 38 and 42 of sidewall 28 and portions 46, 50, 54 and 58 of sidewall 44 could be configured to assist in or facilitate the conversion of deformable member from the unexpanded configuration to the expanded configuration. In the illustrated form, portions 30, 34, 38 and 42 of sidewall 28 and portions 46, 50, 54 and 58 of sidewall 44 are provided with a pre-arranged orientation relative to the remaining portions of sidewalls 28 and 44 that directs the manner in which member 14g is deformed as axial compression is applied thereto. However, it should also be appreciated that one or more portions of sidewalls 28 and 44 may be formed from a material that has greater deformability properties relative to the remaining portions of sidewalls 28 and 44 in order to direct the manner in which member 14g is deformed upon the application of sufficient axial compression. Still, in other forms, it is contemplated that one or more portions of sidewalls 28 and 44 could be provided with unique sizes, shapes or surfaces features, including but not limited to notches, cut, divots and/or grooves, in order to direct the manner in which member 14g is deformed upon the application of sufficient axial compression.

With reference to FIG. 3A, each of members 14a-14g in the expanded configuration is generally elongated in a direction that extends transversely to elongated shaft 60. In addition, once expanded, each of members 14a-14g is positioned on second portion 70 of elongated shaft 60. In this arrangement, compression member 76 can engage with threading 72 on second portion 70 to apply axial compression to members 14a-14g and to prevent proximal movement of deformable members 14a-14g. Similarly, once engaged with threading 72 on second portion 70, compression member 76 may also lock deformable members 14a-14g in the expanded configuration. After compression member 76 has been positioned on second portion 70, first portion 68 can be rotated relative to second portion 70 in order to sever first portion 68 from second portion 70 at frangible portion 74. Moreover, in one or more forms it is contemplated that implant 10 could be configured to prevent disengagement of compression member 76 from second portion 70. For example, in one form, a pin or other locking member could be positioned through compression member 76 and section portion 70 to prevent further rotation of compression member 76 relative to second portion 70. In another form, a pin or other locking member could be positioned through second portion 70 proximally and adjacent to compression 76 in order to prevent proximal movement of compression member 76. Still, it is also contemplated that compression member 76 could be in the form of a lock nut and/or that second portion 70 could be splayed or otherwise expanded adjacent frangible portion 74 after first portion 68 has been severed in order to prevent proximal movement of compression member 76 from second portion 70.

In the expanded configuration, each of deformable members 14a-14g engages with one or more deformable members in addition to those it engages with in the unexpanded configuration. More particularly, as illustrated in FIGS. 3A and 3C for example, in the expanded configuration deformable member 14g engages with deformable member 14e in addition to deformable member 14f with which it engages in the unexpanded configuration. As another example, in the expanded configuration deformable member 14f engages with deformable member 14d in addition to deformable members 14e and 14g with which it engages in the unexpanded configuration. In another example, in the expanded configuration deformable member 14e engages with deformable members 14c and 14g in addition to deformable members 14d and 14f with which it engages in the unexpanded configuration. Similarly, in the expanded configuration each of deformable members 14a-14g engages with any immediately adjacent deformable member as well as any deformable member(s) positioned on the other side of the immediately adjacent deformable member(s).

Further, the engagement of deformable members 14a-14g with any deformable member positioned on the other side of an immediately adjacent deformable member occurs laterally away from and on opposite sides of elongated shaft 60. For example, in the illustrated form deformable members 14a, 14c, 14e and 14g engage in a side to side arrangement with one another at locations laterally offset from and positioned on opposite sides of elongated shaft 60. Moreover, deformable members 14b, 14d and 14f also engage in side to side arrangement with one another at locations laterally offset from and positioned on opposite sides of elongated shaft 60. In one aspect, the arrangement of deformable members 14a, 14c, 14e and 14g in their expanded configuration where they engage with one another and the arrangement of deformable members 14b, 14d and 14f in their expanded configuration where they engage with one another may increase stability of implant 10 in a direction along elongated shaft 60.

In addition to the foregoing, in the expanded configuration each of deformable members 14a-14g is axially offset about elongated shaft 60 relative to any adjacent member. More particularly, in the illustrated form deformable members 14a, 14c, 14e and 14g are rotated about ninety degrees around elongated shaft 60 relative to deformable members 14b, 14d and 14f, although different values for the axial offset between deformable members 14a, 14c, 14e and 14g relative to deformable members 14b, 14d and 14f are also contemplated. In addition, since deformable members 14a-14g are coupled together in an arrangement that prevents rotational movement of the deformable members about elongated shaft 60 relative to one another, the configuration illustrated in FIGS. 3A-3C is maintained following implantation of implant 10. Moreover, as shown in FIG. 3B, this arrangement also provides implant 10 with an “x” or cross shaped configuration when deformable members 14a-14g are in the expanded configuration. In this configuration, the enlarged intermediate portions of the sidewalls of deformable members 14a-14g provide a lip or edge that can engage with opposing surfaces or endplates of the adjacent bones or bony structures in order to prevent or resist rotation of implant 10 relative to the adjacent bones on bony structures between which it is positioned.

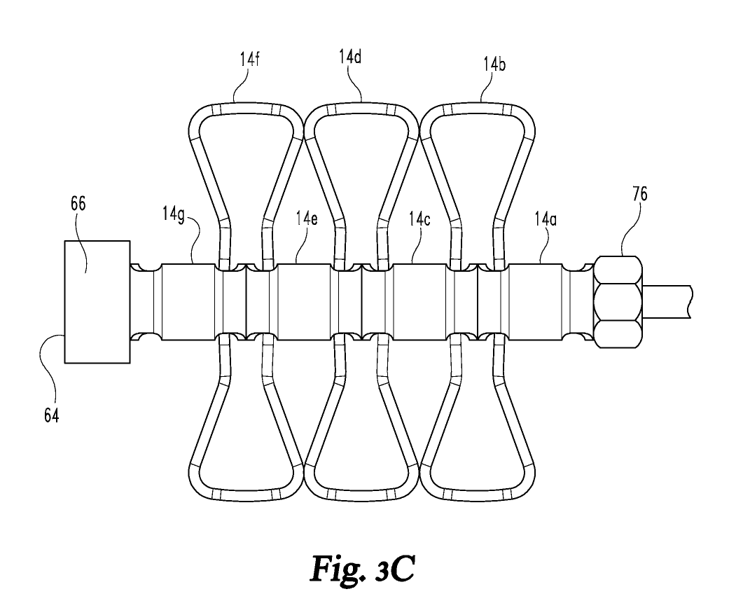

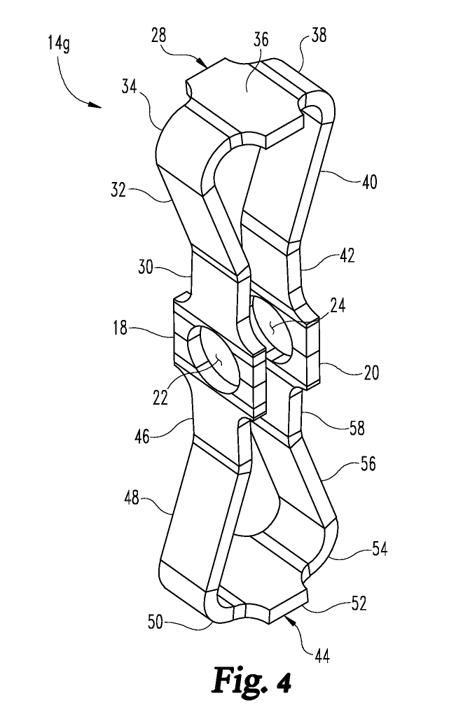

An alternative embodiment implant 110 is illustrated in FIGS. 5 and 6, where like numerals refer to like features of implant 10 previously described. Implant 110 includes a plurality of spacers 178a-178g that are positioned on and configured to freely slide along elongated shaft 60. One of spacers 178a-178g is positioned between the proximal and distal ends of each of deformable members 14a-14g. Similarly, as deformable members 14a-14g are axially compressed and transitioned to the expanded configuration, spacers 178a-178g are moved along elongated shaft 60 and limit the amount which the proximal and distal ends of each one of deformable members 14a-14g can be moved toward each other along elongated shaft 60. Similarly, in contrast to the arrangement of implant 10, the proximal and distal ends of each of deformable members 14a-14g in implant 110 can not be positioned against one another. Accordingly, as the proximal and distal ends of each of deformable members 14a-14g comes into contact with one of spacers 178a-178g positioned therebetween, additional axial compression of deformable members 14a-14g will be prevented.

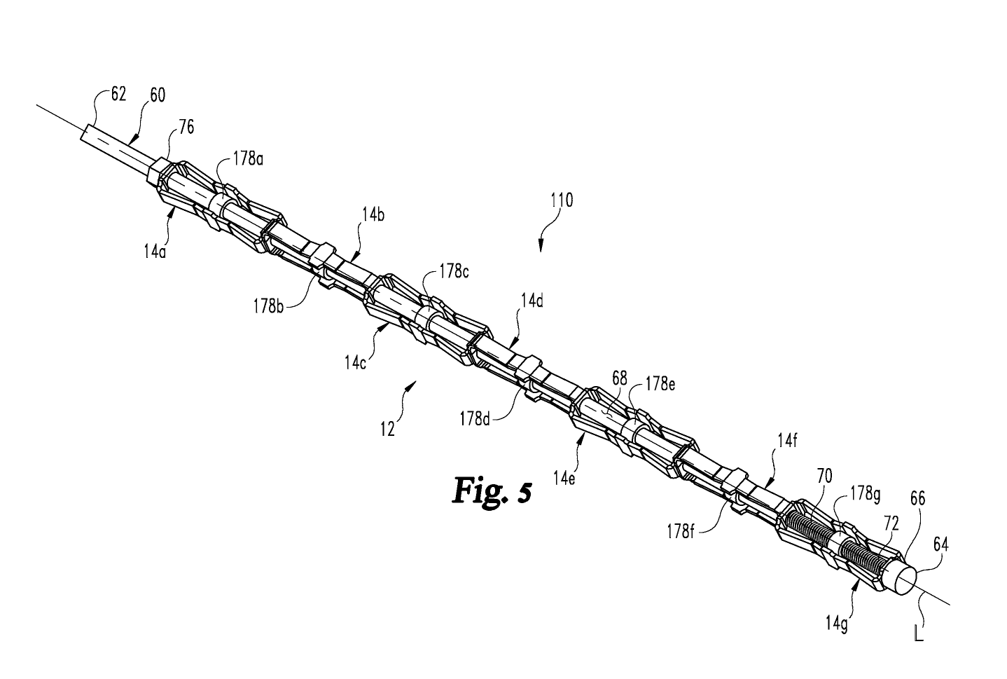

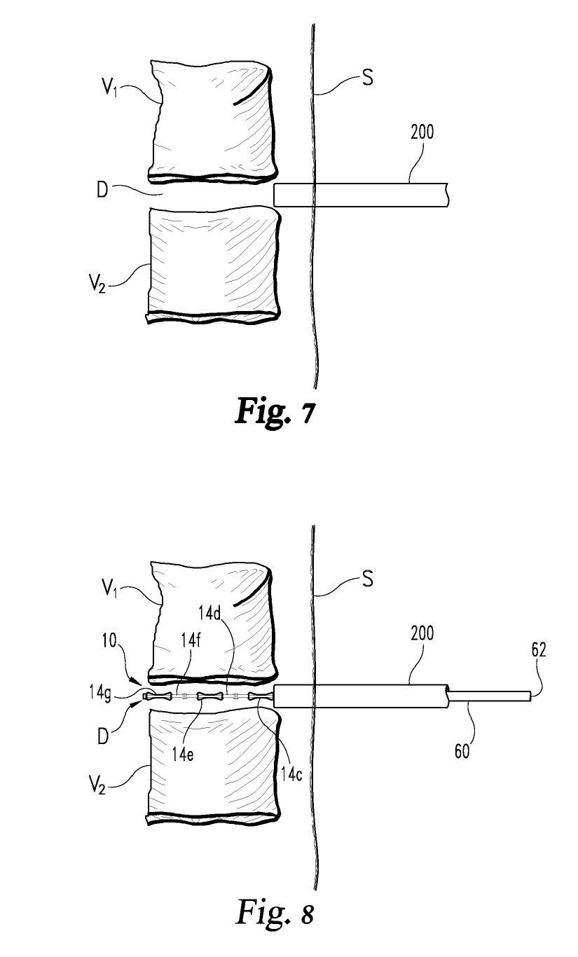

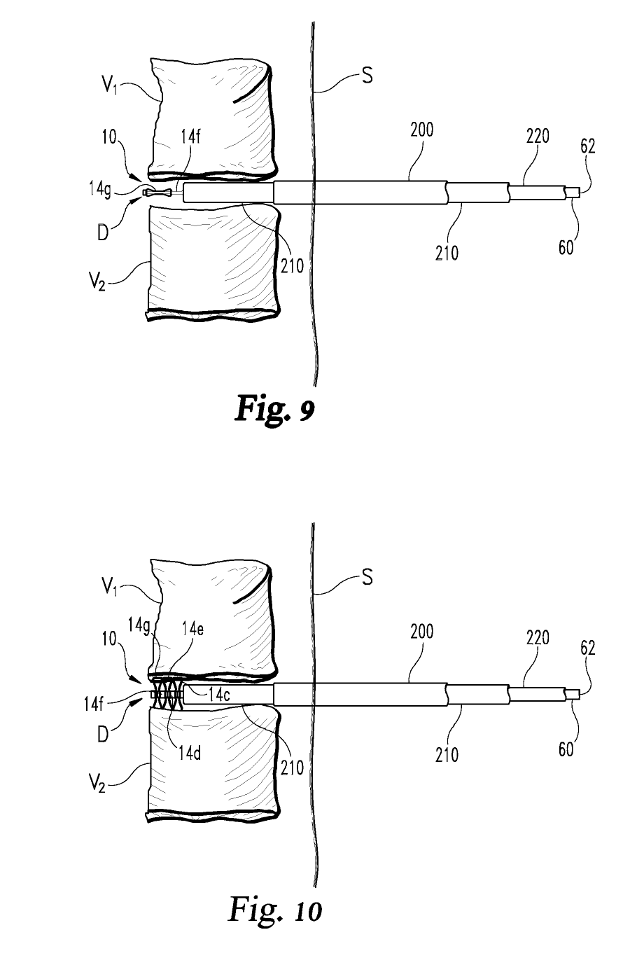

Referring now generally to FIGS. 7-11, further details with respect to one manner for positioning implant 10 between adjacent bony structures are provided. It should be appreciated that implant 110 may be positioned in this or a similar manner as well. In addition, while implant 10 is described as being positioned between adjacent vertebral bodies V₁ and V₂, it should be appreciated that use of implant 10 or implant 110 at other anatomical locations besides the spinal column are contemplated. Furthermore, while the described manner for positioning implant 10 utilizes minimally invasive surgical techniques, it should be appreciated that implant 10 or implant 110 may also be positioned or implanted in a non-minimally invasive approach.

With reference to FIG. 7 for example, a cannula 200 is inserted through an incision in the skin S and advanced to a location adjacent disc space D between adjacent vertebral bodies V₁ and V₂. It should be appreciated that disc space D can be accessed from any of an anterior, posterior, antero-lateral, postero-lateral or lateral approach. In one form, prior to insertion of cannula 200, the skin and tissue can be sequentially dilated via a dilation instrument set (not illustrated) which can include guidewires and/or one or more tissue dilators of increasing size. The tissue dilators are inserted one over another to form a pathway through the skin and tissue to the surgical site in the patient. In such procedures, cannula 200 is positioned over the last inserted dilator to form a pathway through the skin and tissue adjacent to disc space D, and the guidewires and dilators, if used, are removed from cannula 200.

In addition, while not illustrated it should be appreciated that viewing of the surgical site at the end of cannula 200 can be accomplished with viewing instruments mounted on cannula 200, positioned over cannula 200, positioned in other portals in the body, and/or through a viewing system such as lateral fluoroscopy. It is further contemplated that other instruments can be mounted on or used in combination with cannula 200, including but not limited to nerve root retractors, tissue retractors, forceps, cutters, drills, scrapers, reamers, separators, rongeurs, taps, cauterization instruments, irrigation and/or aspiration instruments, illumination instruments, inserter instruments, and the like. Once cannula 200 has been positioned adjacent disc space D, all or part of the spinal disc material positioned between vertebral bodies V₁ and V₂ can be removed from disc space D in order to create an area for implant 10 to be positioned. In addition, one or more sections of one or both of vertebral bodies V₁ and V₂ may be reamed or scraped in order to cause bleeding to facilitate or enhance fusion between the adjacent vertebral bodies V₁ and V₂.

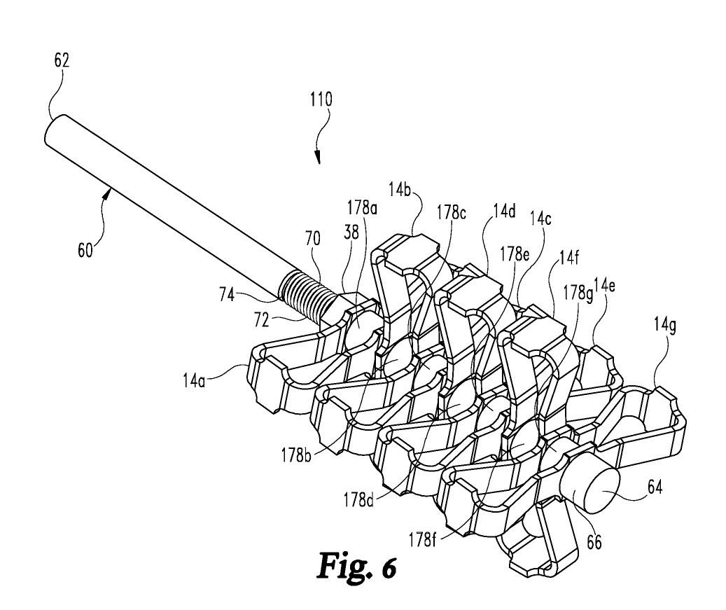

As illustrated in FIG. 8, implant 10 is positioned through cannula 200 such that one or more of deformable members 14a-14g is positioned in disc space D and proximal end 62 of elongated shaft 60 extends from the proximal end of cannula 200. In order to control the order of expansion of deformable members 14a-14g, a sleeve member 210 is positioned in cannula 200 over elongated shaft 60 of implant 10 and advanced distally until its distal end is positioned proximally of one or more the deformable members in disc space D. In addition, a drive member 220 is also positioned in cannula 200 over elongated shaft 60 of implant 10 and within sleeve member 210. Drive member 220 is configured to engage with compression member 76 to advance compression member 76 distally in order to apply axial compression to deformable members 14a-14g. As axial compression is applied, the deformable members 14a-14g which are not positioned within sleeve member 210 are expanded to the expanded configuration. Similarly, it should be appreciated that sleeve member 210 can be proximally moved along elongated shaft 60 such that deformable members 14a-14g are sequentially expanded in a distal to proximal orientation. In this configuration, as distal ones of deformable members 14a-14g are expanded, proximal ones of deformable members 14a-14g which may not initially be positioned within disc space D can be moved distally into disc space D to a location distal of the distal end of sleeve member 210 where they can be expanded to their expanded configuration. Similarly, amongst other things, the ability to control the order in which deformable members 14a-14g are expanded may allow a surgeon positioning implant 10 in disc space D to prevent the expansion of deformable members 14a-14g at a location outside of disc space D.

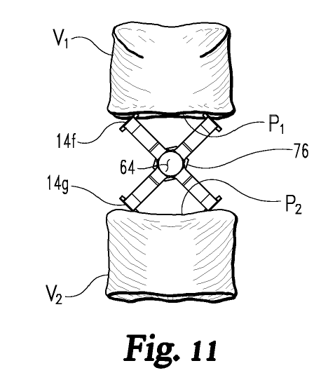

While not previously discussed, it should be appreciated that deformable members 14a-14g can provide distraction to disc space D as they are expanded to the expanded configuration. Alternatively, it is also contemplated that one or more other instruments may be used to provide distraction of disc space D before implant 10 is positioned therebetween. Once deformable members 14a-14g are expanded to the expanded configuration, implant 10 can be moved around disc space D to a desired location. Moreover, implant 10 can also be rotated relative to vertebral bodies V₁ and V₂ to the orientation illustrated in FIG. 11 where each of deformable members 14a-14g extends obliquely to and engages with the endplates Pᵢ and P₂ of vertebral bodies V₁ and V₂, respectively. After implant 10 is positioned at a desired location in disc space D, sleeve member 210 and drive member 220 can be removed from cannula 200. In addition, first portion 68 of elongated shaft 60 can then also be severed or released from second portion 70 such that implant 10 left in disc space D post surgery includes deformable members 14a-14g, second portion 70 of elongated shaft 60 and compression member 76.

The implants disclosed and described herein can be formed from any biocompatible material, including but not limited to non-reinforced polymers, carbon-reinforced polymer composites, PEEK and PEEK composites, shape-memory alloys, titanium, titanium alloys, cobalt chrome alloys, stainless steel, ceramics and combinations thereof and others as well. In addition, while not previously described, it should be appreciated that any suitable osteogenic material or composition is contemplated for placement within and/or use with the devices disclosed in this document. Such osteogenic material includes, for example, autograft, allograft, xenograft, demineralized bone, synthetic and natural bone graft substitutes, such as bioceramics and polymers, and osteoinductive factors, just to provide a few examples. Moreover, the osteogenetic material or composition can also include an effective amount of a bone morphogenetic protein, transforming growth factor β1, insulin-like growth factor 1, platelet-derived growth factor, fibroblast growth factor, LIM mineralization protein (LMP), and combinations thereof or other therapeutic or infection resistant agents.

Alternative configurations and uses of the devices described herein are also contemplated. For example, in one or more forms the devices described herein can be positioned at two or more vertebral levels of the spinal column. In addition, the devices and methods described herein may also be used in surgical procedures involving animals, or in demonstrations for training, education, marketing, sales and/or advertising purposes. In addition, the devices and methods described herein may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. An implant, comprising a body including a plurality of deformable members positionable between an unexpanded configuration and an expanded configuration in response to axial compression, wherein at least a first member of said plurality of deformable members engages with a second member of said plurality of deformable members when each of said first and second members is in said unexpanded configuration and said first member further engages with a third member of said plurality of deformable members when each of said first, second and third members is in said expanded configuration, said second member being positioned between said first member and said third member.

2. The implant of claim 1, wherein in said unexpanded configuration said plurality of deformable members extend in end-to-end relationship with one another along a longitudinal axis.

3. The implant of claim 2, wherein in said expanded configuration said plurality of deformable members are elongated in a direction that extends transversely to said longitudinal axis.

4. The implant of claim 2, wherein said first member engages with said third member on opposite sides of said longitudinal axis when each of said first, second and third members is in said expanded configuration.

5. The implant of claim 4, further comprising an elongated shaft extending through a pair of oppositely positioned apertures on each of said plurality of deformable members.

6. The implant of claim 5, wherein said elongated shaft extends along a longitudinal axis between a proximal end and a distal end and includes a first portion including said proximal end and a second portion including said distal end, said first portion being releasably coupled with said second portion.

7. The implant of claim 6, wherein said second portion of said elongated shaft includes external threading.

8. The implant of claim 6, wherein in said expanded configuration each of said plurality of deformable members is positioned on said second portion of said elongated shaft.

9. The implant of claim 6, further comprising a compression member positioned on said elongated shaft between said proximal end and said plurality of deformable members, said compression member being configured to provide axial compression to said plurality of deformable members to position said plurality of deformable members from said unexpanded configuration to said expanded configuration.

10. A method, comprising positioning an implant according to claim 1 in a disc space between an upper vertebra and a lower vertebra and applying axial compression to said plurality of deformable members to position said plurality of deformable members in said expanded configuration.

11. The method of claim 10, wherein in said expanded configuration alternating ones of said plurality of deformable members extend transversely to one another and each of said plurality of deformable members engages with said upper vertebra and said lower vertebra.

12. An implant, comprising:
an elongated shaft;
a plurality of deformable members positioned about said elongated shaft, each of said plurality of deformable members including a body positionable between an unexpanded configuration and an expanded configuration, said body including oppositely positioned first and second ends and first and second sidewalls extending between said first and second ends; and
wherein in said unexpanded configuration said body of each of said plurality of deformable members is elongated along said elongated shaft, and in said expanded configuration said body of each of said plurality of deformable members extends transversely to said elongated shaft and oppositely positioned first and second portions of each of said first and second sidewalls extend obliquely away from said elongated shaft and one another.

13. The implant of claim 12, wherein each of said first and second sidewalls further includes an intermediate portion positioned between said first and second portions.

14. The implant of claim 13, wherein when said body is in said expanded configuration an arcuate portion extends between each of said first and second portions and said intermediate portion.

15. The implant of claim 13, wherein said intermediate portion extends laterally beyond said first and second portions.

16. The implant of claim 12, wherein said elongated shaft extends through an aperture on each of said first and second ends of said body of each of said plurality of deformable members.

17. The implant of claim 12, wherein said plurality of deformable members are non-rotatably arranged relative to one another.

18. The implant of claim 17, wherein said first and second sidewalls of said body of each of said plurality of deformable members are axially offset about said elongated shaft relative to said first and second sidewalls of said body of any adjacent deformable member.

19. The implant of claim 12, further comprising a plurality of spacer members, wherein one of said plurality of spacer members is positioned between said first and second ends of said body of each of said plurality of deformable members.
